SEQUENCE LISTING (10 sequences)

SEQ ID NO: 1 - DNA, 27 bp, Homo sapiens
  misc_feature (1)..(27)
ttctcaaccaagaacctggacctgagc

SEQ ID NO: 2 - DNA, 27 bp, Homo sapiens
  misc_feature (1)..(27)
tgctagatttgtctccacagccaccag

SEQ ID NO: 3 - DNA, 19 bp, Homo sapiens
  misc_feature (1)..(19)
caatgcctcctgcaccacc

SEQ ID NO: 4 - DNA, 19 bp, Homo sapiens
  misc_feature (1)..(19)
ggtggcagtgatggcatgg

SEQ ID NO: 5 - DNA, 21 bp, Homo sapiens
  misc_feature (1)..(21)
tgcagttttgccaaggagtgc

SEQ ID NO: 6 - DNA, 21 bp, Homo sapiens
  misc_feature (1)..(21)
cagacagagctctcttccatc

SEQ ID NO: 7 - DNA, 20 bp, Homo sapiens
  misc_feature (1)..(20)
gagtgacaagcctgtagccc

SEQ ID NO: 8 - DNA, 20 bp, Homo sapiens
  misc_feature (1)..(20)
ccttgaagaggacctgggag

SEQ ID NO: 9 - DNA, 19 bp, Homo sapiens
  misc_feature (1)..(19)
tcagccagcaccctggagc

SEQ ID NO: 10 - DNA, 24 bp, Homo sapiens
  misc_feature (1)..(24)
gccaggatccattttcttcttcac

(19) United States
(12) Patent Application Publication
(10) Pub. No.: US 2018/0326089 A1
(43) Pub. Date: Nov. 15, 2018

(54) PROTEIN NANOSTRUCTURE BASED DRUG DELIVERY SYSTEM FOR THE DELIVERY OF THERAPEUTIC AGENTS TO THE ANTERIOR SEGMENT OF THE EYE

(71) Applicant: Council of Scientific & Industrial Research, New Delhi (IN)

(72) Inventors: Mohammad Ahsan SAAD, Hyderabad, Andhra Pradesh (IN); Mohan Rao CHINTALAGIRI, Hyderabad, Andhra Pradesh (IN)

(21) Appl. No.: 15/776,765

(22) PCT Filed: Nov. 16, 2016
(86) PCT No.: PCT/IN2016/050400

§ 371 Date: May 16, 2018

(30) Foreign Application Priority Data
Nov. 17, 2015 (IN) 3745/DEL/2015

(51) Int. Cl. A61K 47/69; A61K 47/68; A61K 47/61; A61K 31/496; A61P 27/02

(52) U.S. Cl. CPC A61K 47/6931; A61K 47/6849; A61P 27/02; A61K 31/496; A61K 47/61

(57) ABSTRACT

A multifunctional system in which a nanostructure (size range of about 10-1000 nm) degrades on exposure to an infection and its associated inflammatory milieu. The degraded nanostructures release the encapsulated drug during the process of degradation, where the kinetics of drug release is determined by the severity of the infection and inflammation. This degradation is triggered by proteases secreted by the pathogen, host polymorphonuclear leucocytes and other host cells. The nanostructures are conjugated to anti-TLR (Toll-like receptor) ligands for targeting the corneal epithelium and blocking the inflammatory pathway.

### FIELD OF INVENTION

The present invention provides nanostructure based drug delivery system for therapeutic applications. The present invention provides nanostructure based drug delivery system wherein nanostructure loaded with an anti-microbial drug or an anti-inflammatory therapeutic agent, the release of which is regulated by the degradation of the nanostructure.

### BACKGROUND OF THE INVENTION

The corneal tissue comprises mainly of a multilayered epithelium followed by a layer of densely packed collagen bundles called the stroma and an innermost single cell endothelial layer. The stromal layer consists of relatively few cells, mainly the collagen secreting keratocytes and unevenly distributed immune cells (dendritic cells, langerhans cells, B- and T-lymphocytes) mostly in the peripheral cornea and relatively less in the centre. Apart from the immune cells in the corneal stroma, the conjunctiva associated lymphoid tissue (CALT) and the lacrimal drainage associated lymphoid tissue (LDALT), together constitute the eye associated lymphoid tissue (EALT). These lymphoid tissues can detect corneal antigens and prime respective effector cells for a response. (Knop and Knop 2005a; Knop and Knop 2005b; Vantrappen et al. 1985; and Akpek and Gottsch 2003.)

The corneal epithelium like other surface tissues is continuously exposed to a wide range of foreign matter including pathogenic micro-organisms and provides the first line of host defense system. The corneal innate immune system comprises a series of pattern recognition receptors (PRRs), the toll-like receptors (TLRs) which play a key role in pathogen detection and eliciting a host immune response at the corneal surface. Recognition of pathogen associated molecular patterns (PAMPs) by TLRs activates a series of signaling events leading to an up-regulation in the expression of pro-inflammatory cytokines (IL-1β, TNFα, IL-6, etc), chemotactic molecules (MIP family proteins, IL-8, etc), prostaglandins (PGE2), histamine, leukotrienes, as well as proteases (MMPs, serine proteases, etc). (Klotz et al. 2000; Zhao and Wu 2008a; Zhao and Wu 2008b; Ibeagha-Awemu et al. 2008; Johnson and Pearlman 2005; Johnson et al. 2005; Kumar and Yu 2006; Song et al. 2007; Kumar et al. 2006; Ren and Wu 2011; Fukata et al. 2006 and Madianos et al. 2005.)

Although well protected (by the eyelids, the eye socket, tears, and the sclera), the cornea may be damaged by foreign objects which may lead to infection and inflammation, a condition commonly referred to as keratitis. While sterile keratitis is caused by corneal injury, alkali burns or prolonged use of contact lenses, infectious keratitis can be caused by a variety of pathogens like fungi, bacteria, viruses, protozoans and other parasites. The incidence of keratitis has been increasing due to the increase in use of contact lenses, wide-spread use of broad-spectrum antibiotics and steroids, medical interventions at the anterior segment, etc. Keratitis is one of the leading causes of corneal blindness, and an early diagnosis and treatment can help in preventing complications such as hypopyon formation, endopthalamitis, blindness or even enucleation of the eye ball. (Sharma 2001; Srinivasan et al. 2008; Gao et al. 2011 and Guo et al. 2012.)

Treatments for corneal infections require frequent administration of antibiotics and anti-inflammatory drugs. Topically applied antibiotics require high dosage frequency due to pre-corneal clearance, whereas application of anti-inflammatory drugs requires precise control over dosage, as a high dose may exacerbate the infection while a low dose may exaggerate the host response and lead to inflammation mediated corneal damage. Such an intensive medication course lacks patient compliance and often needs hospitalization.

Designing a successful system for keratitis requires is challenging. The two major requirements are maintaining a therapeutic concentration of antibiotics in the corneal tissue and keeping a precise control over the anti-inflammatory drug levels in the cornea. (Gokhale 2008; Wilhelmus 2002; Dajcs et al. 2004 and Thibodeaux et al. 2004.)

Structures formed by molecules in the size range of 1 nm to 100 nm are commonly referred to as nanostructures. Nanostructures have been projected to have an enormous potential in drug delivery. Nanostructures have gained popularity in recent times owing to their ability to deliver drugs to various organs, under different pathological conditions viz. drug delivery to tumors, ocular drug delivery, drug delivery to the central nervous system (CNS), pulmonary drug delivery, etc. Drug delivery through nanostructures has several advantages viz. reducing toxic side effects, higher targeting efficiency and incorporation of multiple components thereby making the nanostructure multifunctional. (Wang et al. 2011; Kompella et al. 2010; Patel et al. 2012; Salvati et al. 2013 and Bailey and Berkland 2009.)

Nanostructures have offered new approaches to the delivery of therapeutic agents to the anterior segment of the eye. Delivery systems for topical administration to the eye include nanostructures, liposomes, suspensions, gels, erodible and non-erodible inserts and rods. Timoptol®-LA, Ocusert®, Carbopol®, etc are are examples of FDA-approved nanostructure-based therapeutics for ocular drug delivery. However, despite the advances in ocular drug delivery, an efficient system for corneal infections and inflammation (keratitis) is yet to be developed. (Conway 2008.)

Gelatin has been used extensively in food and drug industry. Moreover the scleral layer of the cornea is composed of collagen, the sole source of gelatin. Being a protein it also provides various functional groups for chemical modifications and cross-linking. Being a derivative of collagen, gelatin contains abundant RGD (Arginine-Glycine-Aspartate) repeats which have been shown to help in epithelial cell binding and proliferation. (Haurowitz et al. 1943; Ofokansi et al. 2010; Pierschbacher and Ruoslahti 1984; Pierschbacher and Ruoslahti 1984b and Lu et al. 2009.)

Keratitis due to fungal infections is more common in tropical countries like IndiaKetoconazole is an imidazole derivative with broad-spectrum, fungistatic activity and is active against a variety of fungal strains including Aspergillus species, Candida species and some Fusarium species. It has been recommended topically and systemically for treating fungal keratitis. Ketoconazole is a poorly water soluble drug (Ghannoum and Rice 1999 and Zhang et al. 2008.)

Cyclodextrins are oligosaccharides with a hollow lipophilic central cavity and a hydrophilic outer surface. Hydrophobic drugs can be complexed with cyclodextrins by getting buried in the lipophilic cavity thereby forming water-soluble complexes. Cyclodextrins have been extensively used in eye drop formulations of lipophilic drugs, such as steroids and some carbonic anhydrase inhibitors. Apart from increasing the aqueous solubility, cyclodextrins are also known to enhance drug absorption into the eye and reduce local irritation. (Loftsson and Stefansson 2002.)

As the pathogen binds to the corneal epithelium it triggers a host response via the TLR pathway. The host response includes the secretion of pro-inflammatory cytokines.

These pro-inflammatory cytokines recruit polymorphonuclear leucocytes (PMN). The combined secretions of the epithelial cells and PMNs have an anti-microbial effect. MMPs, particularly gelatinases A and B (MMP-2 and -9), are a key component in the downstream of the TLR signalling pathway and help in PMN infiltration through extra-cellular matrix. In some cases, there may be an exaggerated immune response, leading to irreversible tissue destruction. This may result in degradation and disorganization of the extracellular matrix and scleral layer of the cornea, and eventually compromised or permanent visual loss. (Akpek and Gottsch 2003.)

Pathogenic fungi are known to secrete a variety of proteases such as aminopeptidases, carboxypeptidases and dipeptidyl-peptidases. In fact, pathogenic fungi are known to secrete proteases depending on the substrate they are grown on, thereby deriving essential nutrients for their survival. They are known to secrete collagenases when present at the corneal surface which leads to the degradation of the scleral collagen. Levels of host proteases are also increased during the inflammatory response. (Monod et al. 2002.)

Because both microbial and host proteases are extremely efficient in hydrolyzing gelatin (denatured collagen), a nanostructure of about 100-200 nm was engineered with a core composed of gelatin. Gelatin is a biocompatible and biodegradable polymer. Moreover, collagen, the native protein from which gelatin is derived, is present in the eye, more specifically in the stroma, the middle cell layer of the cornea, and has been extensively employed in ocular applications. Gelatin matrix has proven to be a good carrier system for hydrophilic drugs and oligonucleotides. Hydrophobic drugs can be encapsulated in gelatin nanostructures after forming soluble inclusion complexes with cyclodextrins. (Vandervoort and Ludwig 2004.)

The use of anti-inflammatory drugs in the treatment of keratitis is to dampen local inflammation. Steroidal anti-inflammatory drugs help to resolve corneal inflammation; facilitate epithelial and stromal healing; and minimize corneal neovascularization. But, the use of steroids is a point of concern as they can exacerbate the infection by suppressing host immunity and may promote recrudescence. An alternative to steroids are non-steroidal anti-inflammatory drugs (NSAIDs). Adverse effects of NSAIDs may include burning, stinging and ocular irritation, or hypersensitivity reactions such as itching, redness and photosensitivity. Although these adverse effects are taken care off while developing NSAIDs based formulations, the use of these drugs without a concomitant use of steroids has been associated with the development of corneal infiltrates. Other NSAIDs related corneal complications include superficial punctate keratitis, epithelial defects, corneal melting and delayed wound healing. (Wilhelmus 2002.)

With the recent emergence of nanoparticle based smart drug delivery systems, developing a vehicle for keratitis therapy seems possible and may eventually prove beneficial. The nanostructures of the present invention thus presents a smart drug delivery system with host controlled anti-microbial and anti-inflammatory effects. (Zhao and Wu 2008a; Zhao and Wu 2008b; Johnson and Pearlman 2005; Johnson et al. 2005; Gao et al. 2011 and Guo et al. 2012.)

### SUMMARY OF THE INVENTION

The main embodiment of the present invention provides a nanostructure based drug delivery system comprising:
(a) gelatin matrix;
(b) anti-TLR4 ligand conjugated to the gelatin matrix surface;
(c) a therapeutic agent;
wherein
   the gelatin matrix is held together by covalent cross-linking through glutaraldehyde; the anti-TLR4 ligand is an anti-TLR4 antibody that is chemically conjugated to the surface of the nanostructure by EDC-NHS chemistry where the primary amine groups of the anti-TLR4 antibody is conjugated to the free —COOH groups in the gelatin matrix; the therapeutic agent is an anti-microbial drug or an anti-inflammatory drug encapsulated in the gelatin matrix.

Another embodiment of the present invention provides a nanostructure based drug delivery system as herein described, wherein the gelatin matrix is made of RGD sequences or a positively charged outer surface by conjugating molecules like poly-lysine, chitosan etc.

Another embodiment of the present invention provides the nanostructure based drug delivery system as herein described, wherein the therapeutic agent is hydrophobic or hydrophilic.

Another embodiment of the present invention provides the nanostructure based drug delivery system as herein described, wherein the hydrophobic therapeutic agent is complexed with a cyclodextrin.

Another embodiment of the present invention provides the nanostructure based drug delivery system as herein described, wherein the cyclodextrin is selected from methyl-β-cyclodextrins, hydroxyl propyl β-cyclodextrins.

Another embodiment of the present invention provides the nanostructure based drug delivery system as herein described, wherein the therapeutic agent is ketoconazole.

Another embodiment of the present invention provides the nanostructure based drug delivery system as herein described, wherein the therapeutic agent is released from the nanostructure through degradation by proteases secreted by the host comprising matrix metallo-proteases or serine proteases and/or proteases secreted by the pathogens.

Another embodiment of the present invention provides the nanostructure based drug delivery system as herein described, wherein the nanostructure is about 10-1000 nm in diameter.

Another embodiment of the present invention provides a method of preparing a nanostructure based drug delivery system as herein described, comprising:
a. preparing a solution of the therapeutic agent;
b. dissolving gelatin matrix in the therapeutic agent;
c. preparing drug loaded gelatin nanoparticles using double desolvation method;
d. conjugating anti-TLR4 ligand to the nanoparticles obtained in step (c) using carbodiimide method.

Another embodiment of the present invention provides a method of preparing a nanostructure based drug delivery system as herein described, comprising:
a. dissolution of ketoconazole with methyl-β-cyclodextrin;
b. dissolving of gelatin into ketoconazole-methyl-β-cyclodextrin complex solution obtained in step (a);
c. preparing ketoconazole loaded gelatin nanoparticles using double desolvation method;
d. conjugating anti-TLR4 antibody to the nanoparticles obtained in step (c) using carbodiimide method.

Another embodiment of the present invention provides a method of delivering an antibiotic and/or an anti-inflammatory therapeutic agent to an infection in a subject, wherein the method comprises administering to the subject the nanostructure as herein described. Another embodiment of the present invention provides a method of treating infectious and/or sterile keratitis, wherein the method comprises administering a therapeutically effective amount of the nanostructure as herein described.

Another embodiment of the present invention provides use of the nanostructure based drug delivery system as herein described, for treatment of infections or inflammations.

Another embodiment of the present invention provides use of the nanostructure based drug delivery system as herein described, wherein the infection is infectious and/or sterile keratitis.

Another embodiment of the present invention provides a pharmaceutical composition comprising a nanostructure based drug delivery system as herein described.

Another embodiment of the present invention provides use of the pharmaceutical composition as herein described, for treatment of infections or inflammations.

Another embodiment of the present invention provides use of the pharmaceutical composition as herein described, wherein the infection is infectious and/or sterile keratitis.

Another embodiment of the present invention provides a kit for treatment of infectious or sterile keratitis comprising the nanostructure based drug delivery system herein described and an instruction manual.

### BRIEF DESCRIPTION OF DRAWINGS

FIG. 1: Nanoparticle cartoon.

FIG. 2: Dissolution of ketoconazole by methyl-β-cyclodextrin. (A) Cyclodextrin structure and cartoon showing the outer hydrophilic surface and inner hydrophobic surface. B) Chemical structure of ketoconazole. C) Phase solubility diagram of ketoconazole in the presence of methyl-β-cyclodextrin.

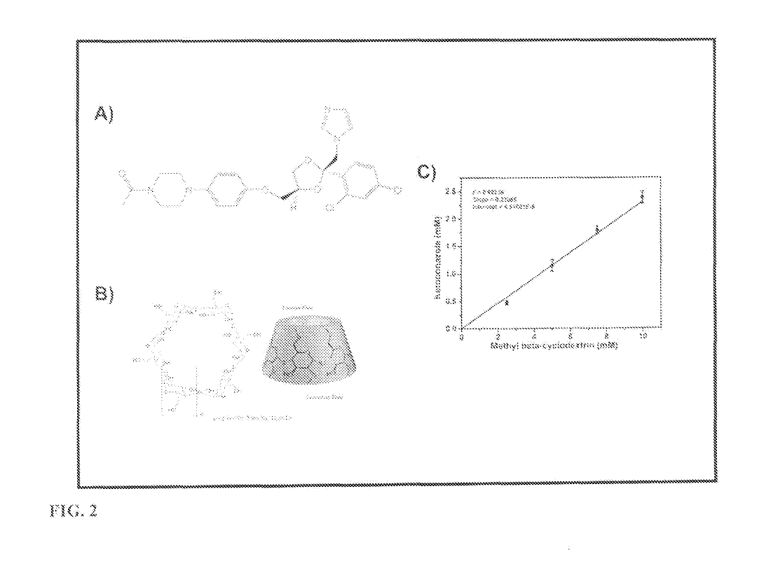

FIG. 3: Anti-TLR4 antibody conjugation to gelatin nanoparticles. (A) Schematic illustration. (B) Nanoparticle size distribution. (C) Anti-TLR4 antibody conjugated nanoparticle size distribution.

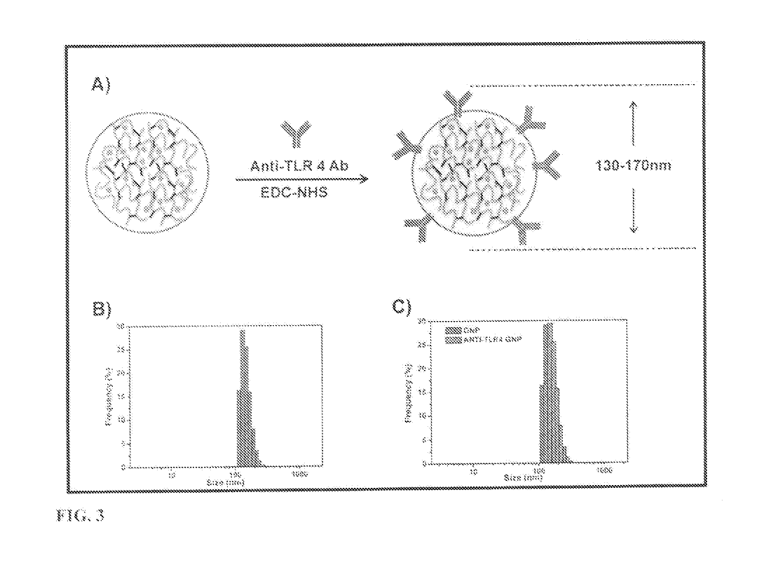

FIG. 4: Surface charge of gelatin nanoparticles before and after antibody conjugation.

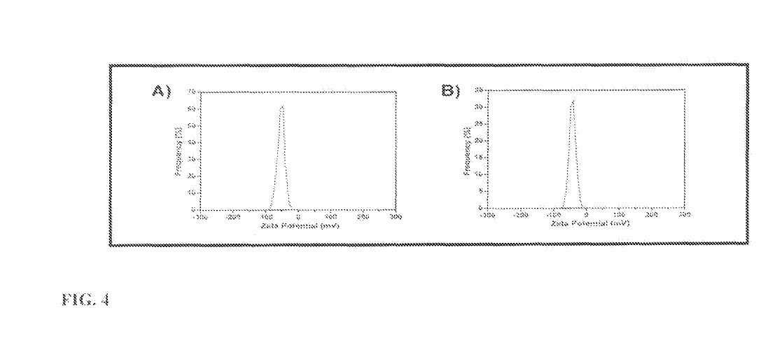

FIG. 5: MIC of ketoconazole, ketoconazole-methyl-β-cyclodextrin complex and ketoconazole-methyl-β-cyclodextrin complex encapsulated in gelatin nanoparticles. Growth percent of Aspergillus flavus at different concentrations of ketoconazole (in μg) (A), ketoconazole-methyl-β-cyclodextrin complex (in mM) (B) and ketoconazole-methyl-β-cyclodextrin complex encapsulated gelatin nanoparticles (mg) (C). Growth percent normalized for ketoconazole concentration in μg for the three formulations

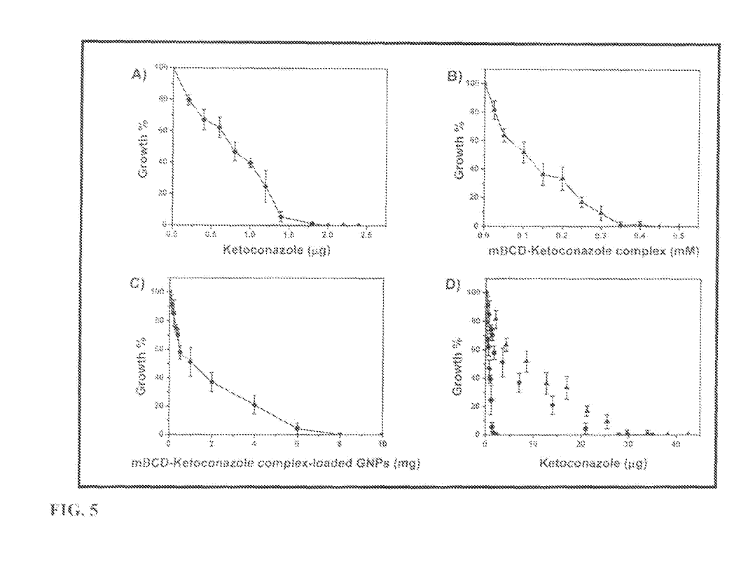

(D). (The minimum concentration at which no growth was observed is referred to as MIC).

FIG. 6: HCE cell interaction with gelatin nanoparticles. A) untreated cells, B) GNP treated cells C) anti-TLR4 conjugated GNP treated cells D) anti-TLR4 conjugated GNP and LPS treated cells.

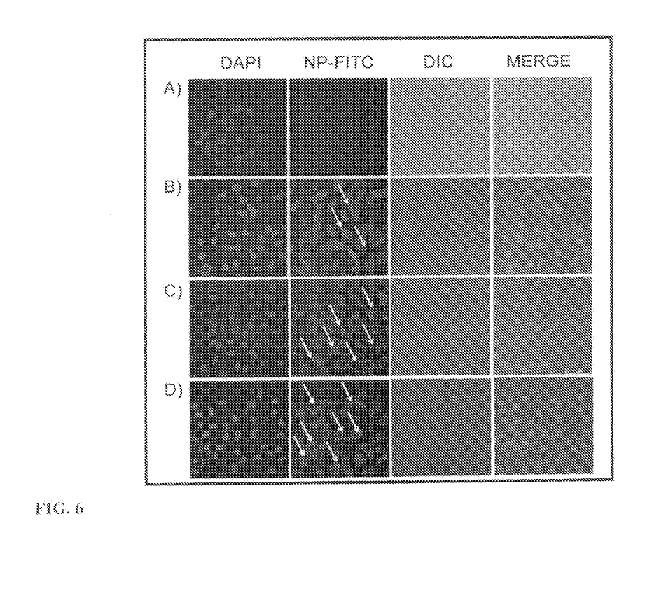

FIG. 7: Flow cytometry for GNP binding to HCE cells as a function of LPS. (A) Untreated cells (blue), (B) bare GNP treated cells (green), (C) anti-TLR4 conjugated GNPs (purple) and (D) anti-TLR4 conjugated GNPs along with LPS (pink). (E) Histogram representation of the cell associated fluorescence in groups A, B, C and D.

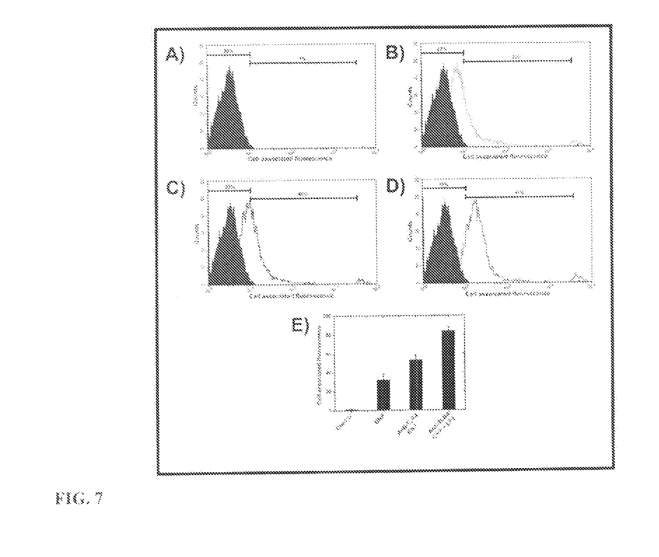

FIG. 8: Histogram for mRNA levels of pro-inflammatory cytokines upon treatment with LPS, LPS with bare GNPs and LPS with anti-TLR4 conjugated GNPs. Histogram for mRNA levels of TLR4 (A), IL-8 (B), TNFα (C) and MMP2 (D) normalized with GAPDH.

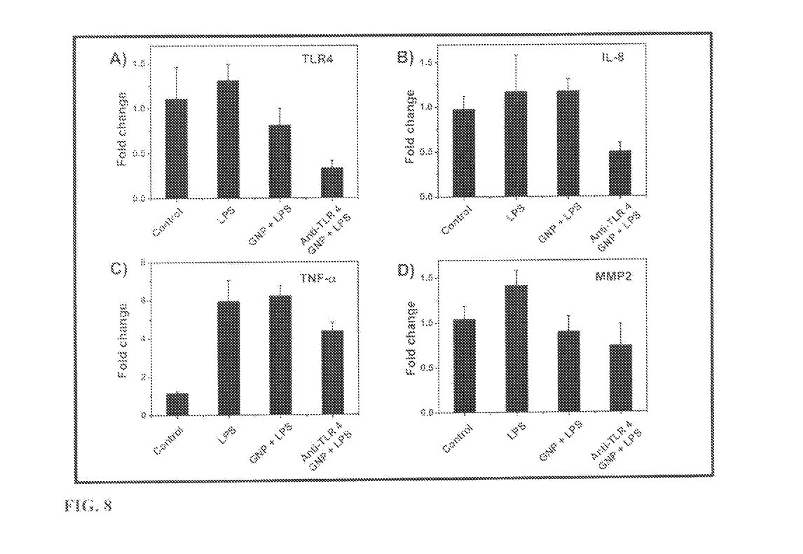

FIG. 9: Drug release by gelatin nanoparticles as a function of protease concentration.

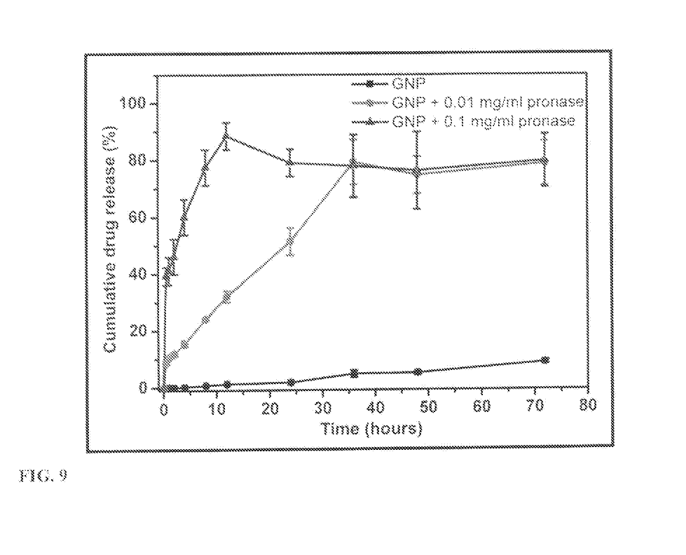

FIG. 10: Clinical micrographs of rat eyes treated with various formulations.

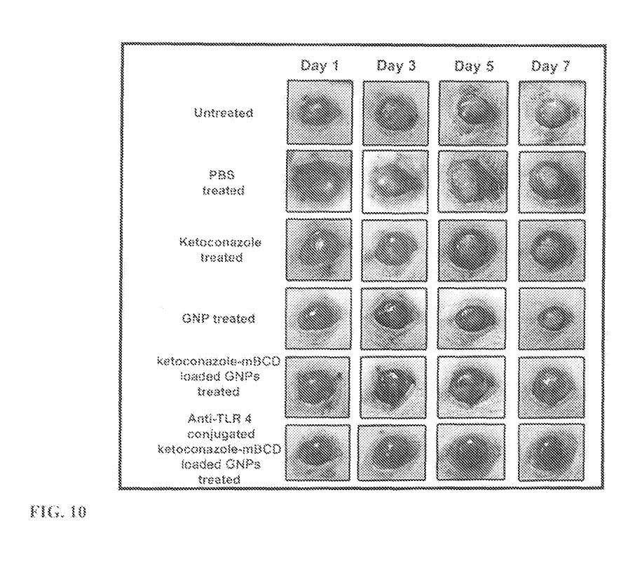

FIG. 11: Hematoxylin and Eosin staining of corneal sections obtained from infected and treated rat eyes. (A) Uninfected control; (B) infected and untreated (samples were taken on day 3 for histopathology studies. Samples collected at day 7 or beyond had a damaged cornea, therefore sectioning and staining was not possible for animals in group 1, 2, 3 and 4). (C) Group 5: administered with ketoconazole-methyl-β-cyclodextrin complex encapsulated gelatin nanoparticle and (D) Group 6: administered with Anti-TLR4 conjugated ketoconazole-methyl-β-cyclodextrin complex encapsulated gelatin nanoparticle.

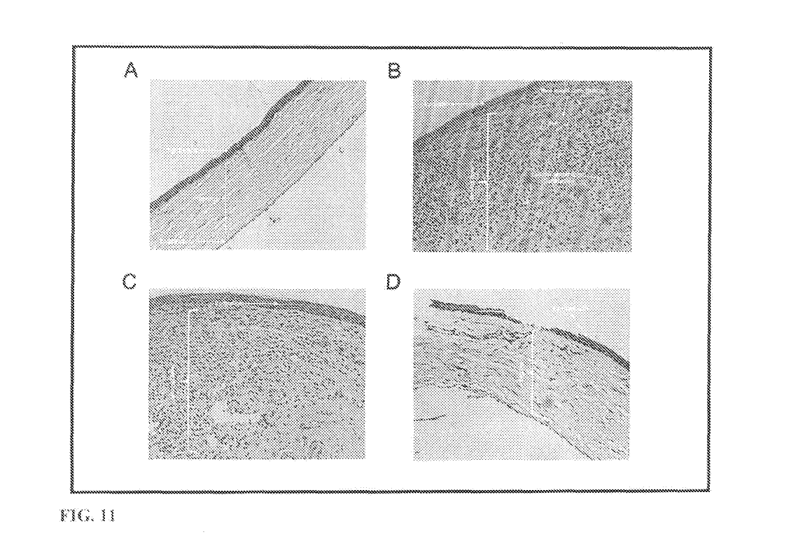

FIG. 12: Eye ball culture for determining fungal load. (A) Uninfected control; (B) Group 1: infected and untreated; (C) Group 2: administered with PBS; (D) Group 3: administered with ketoconazole; (E) Group 4: administered with empty gelatin nanoparticles; (F) Group 5: administered with ketoconazole-methyl-β-cyclodextrin complex encapsulated gelatin nanoparticle and (G) Group 6: administered with anti-TLR4 conjugated ketoconazole-methyl-β-cyclodextrin complex encapsulated gelatin nanoparticle.

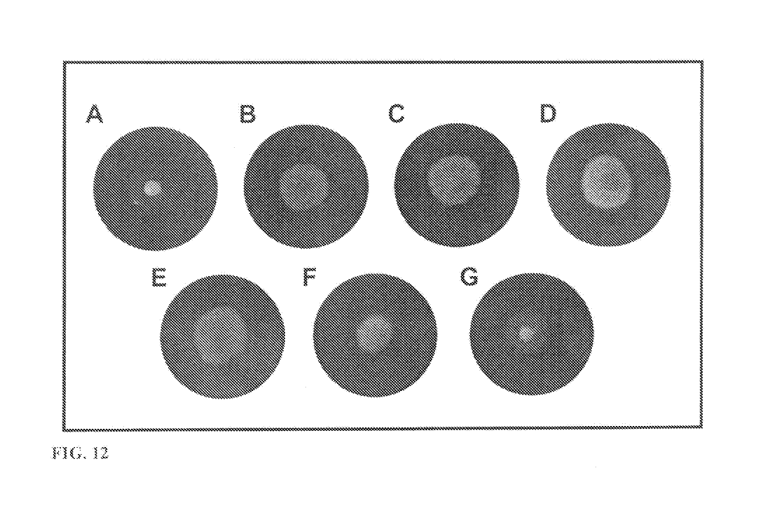

### DETAILED DESCRIPTION OF THE INVENTION

While the invention is susceptible to various modifications and/or alternative processes and/or compositions, specific embodiment thereof has been shown by way of example in the drawings and graphs and will be described in detail below. It should be understood, however that it is not intended to limit the invention to the particular processes and/or compositions disclosed, but on the contrary, the invention is to cover all modifications, equivalents, and alternative falling within the spirit and the scope of the invention as defined by the appended claims.

The graphs, figures and protocols have been represented where appropriate by conventional representations in the drawings, showing only those specific details that are pertinent to understanding the embodiments of the present invention so as not to obscure the disclosure with details that will be readily apparent to those of ordinary skill in the art having benefit of the description herein.

The following description is of exemplary embodiments only and is not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention.

The terms “comprises”, “comprising”, or any other variations thereof, are intended to cover a non-exclusive inclusion, such that one or more processes or composition/s or systems or methods proceeded by “comprises . . . a” does not, without more constraints, preclude the existence of other processes, sub-processes, composition, sub-compositions, minor or major compositions or other elements or other structures or additional processes or compositions or additional elements or additional features or additional characteristics or additional attributes.

In this specification and in the claims that follow, reference will be made to a number of terms that shall be defined to have the following meanings:

It must be noted that, as used in the specification/description and the appended claims and examples, the singular forms “a”, “an” and “the” may include plural referents unless the context clearly dictates otherwise.

Ranges may be expressed herein as from “about” one particular value, and or “to about” another particular value. When such a range is expressed, another aspect includes from the one particular value and or to the other particular value. Similarly, when values are expressed as approximations, by use of the antecedent “about”, it will be understood that the particular value forms another aspect. It will be further understood that the endpoints of each of the ranges are significant both in relation to the other endpoint, and independently of the other endpoint.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The present invention provides a nanostructure based drug delivery system having therapeutic use. More specifically, present invention relates to an efficient nanostructure based drug delivery system for the treatment of infection or inflammation. More specifically, the present invention relates to an efficient nanostructure based drug delivery system for the treatment of keratitis and other ocular drug delivery applications.

An aspect of the present invention provides for a nanostructure based drug delivery system comprising nanostructures that are useful in the treatment of diseases related to corneal infections and inflammation. The term nanostructure may refer to any structure that measures less than about 1000 nm in diameter. The nanostructures of the invention may be useful in the treatment of infections and inflammation. The nanostructure of the invention has the ability to degrade under the pathological condition thereby, releasing the drug. The nanostructure of the invention contains a drug complexed with methyl-β-cyclodextrin dispersed in a matrix of gelatin. The degradation of the gelatin nanostructure by proteases present at the infection site allows for the release of the drug.

In another aspect, the present invention provides an efficient nanostructure based drug delivery system for the delivery of anti-microbial drugs to the site of infection (cornea).

In another aspect, the present invention provides an efficient nanostructure based drug delivery system with an increased residence time on the corneal surface.

In another aspect, the present invention provides a sustained delivery of the encapsulated drug at the site of infection (cornea).

In another aspect, the present invention provides to suppress the host inflammatory response at the site of infection.

In another aspect, the present invention provides that the nanostructure is biodegradable in the conditions of infection and inflammation.

In another aspect, the present invention provides an efficient nanostructure based drug delivery system for the delivery of anti-microbial drugs to the site of infection (cornea).

In another aspect, the present invention provides for the synthesis of nanostructure based drug delivery system for the treatment of keratitis.

In another aspect, the present invention provides for the synthesis of nanostructure based drug delivery system for drug delivery to the anterior segment of the eye.

In another aspect, the present invention provides for the synthesis of nanostructure based drug delivery system for drug delivery to the cornea.

In another aspect, the present invention provides for the synthesis of nanostructures for drug delivery to the anterior segment of the eye infected with microbial pathogens.

In another aspect, the present invention provides for the synthesis of nanostructures for drug delivery to anterior segment of the eye suffering from inflammation either sterile or infectious.

In another aspect, the present invention provides for the synthesis of nanostructures with an increased retention time on the eye/corneal surface.

In another aspect, the present invention provides for the synthesis of nanostructures with conjugated surface ligands for binding to the corneal surface.

In another aspect, the present invention provides for the synthesis of nanostructures with conjugated surface ligands for binding and blocking certain receptors on the corneal epithelial cell surface involved either directly or indirectly in inflammatory pathways.

In another aspect, the present invention provides for the synthesis of nanostructures with conjugated surface ligands for binding and blocking certain receptors on the cell surface which are over expressed under the conditions of infection and/or inflammation and are directly or indirectly involved in inflammatory pathways.

In another aspect, the present invention provides for the synthesis of nanostructures loaded with anti-microbial or anti-inflammatory drugs or both.

In another aspect, the present invention provides for the synthesis of nanostructures, the core of which can degrade in the infection and/or inflammatory microenvironment and release the encapsulated drug.

In another aspect of the present invention, the nanostructure based drug delivery system comprises (a) a gelatin matrix; (b) anti-TLR4 ligand conjugated to the nanostructure surface; (c) a therapeutic agent.

In another aspect of the present invention, the nanostructure synthesis has been carried out in a way that gives the structure stability and efficient drug loading. The structure is held together by covalent cross-linking through glutaraldehyde. Anti-TLR4 antibodies are conjugated to the nanostructure by EDC-NHS chemistry where the primary amine groups of the anti-TLR4 antibody is conjugated to the free —COOH groups in the gelatin matrix.

Another aspect of the present invention provides for the development of a multi-functional nanostructure based drug delivery system in which nanostructures bind to the corneal surface due to the RGD sequences (Arginine-Glycine-Aspartate repeats) and/or surface ligands that bind to TLRs or TLR associated molecules on the corneal epithelial cell surface (these receptors are over-expressed under inflammatory conditions which may or may not be a consequence of the pathogen invasion. The nanostructure degrades by the action of proteases secreted by the pathogen and host, thereby releasing the encapsulated drug. (Zhao and Wu 2008a and Zhao and Wu 2008b.)

To achieve drug release, a large nanostructure should be triggered to release the drug after binding to the corneal epithelial surface. Several nanostructures have been designed to release their contents via a stimulus (light, heat, ultrasound, magnetic field, etc). but their use to date has been limited and not applied to ocular therapies. Topical therapy is necessary to treat keratitis, which is a major cause of corneal morbidity in developing countries. Water mediated hydrolysis, diffusion, or solvent-controlled release mechanisms can achieve topical effects but do not give preferential release under the pathological situation. To attain both topical therapeutic effects and preferential release under the pathological situation, in the present methods and compositions the drug release is triggered using an endogenous host stimulus characteristic of the pathological situation such as high concentrations of proteases secreted by the pathogen and/or the host. (Dvir et al. 2010; Mura et al. 2013 and Zhu et al. 2012.)

In another aspect, the present invention provides a multifunctional nanostructure based drug delivery system in which a nanostructure degrades on exposure to an infection and its associated inflammatory milieu. The nanostructure releases the encapsulated drug during the process of degradation and the kinetics of drug release is determined by the severity of the infection or tissue damage and associated inflammation. This degradation is triggered by proteases secreted by the pathogen, host polymorphonuclear leucocytes and other host cells.

In another aspect of the invention, the kinetics of drug release is determined by the severity of the infection and inflammation. This degradation is triggered by proteases secreted by the pathogen, host polymorphonuclear leucocytes and other host cells. These conditions make enzymatic degradation of the gelatin nanostructure a highly favourable trigger mechanism for drug release.

In another aspect of the present invention, the particle size, loading of the drug, the rate of release and particle stability are optimized by controlling the reaction conditions for nanoparticle synthesis with slight modifications of the protocol mentioned by Coester et al. 2000.

In yet another aspect of the present invention, the nanostructures are conjugated to anti-TLR (Toll-like receptor) ligands for targeting the corneal epithelium, increasing the corneal residence time of the nanostructure and blocking inflammatory pathways.

In another aspect of the invention, the hallmark property of TLR4 over-expression in corneal epithelial cells under inflammatory conditions has been utilized as a target for gelatin nanoparticles. TLRs are known to be over-expressed under inflammatory conditions and blocking them or down regulating them by siRNA have shown to reduce infection load as well as inflammation.

Yet another aspect of the present invention provides a smart nanostructure based drug delivery system, with a gelatin core/matrix and surface conjugated anti-TLR4 antibodies, which binds and blocks TLR4 associated inflammatory pathway. As the surface expression of TLR4 is dependent on the severity of the infection, so is the particle binding. The particle degradation and hence the drug release is regulated by the protease concentration (secreted by the pathogen and host) at the site of infection.

In an aspect of the present invention, the nanostructure comprises gelatin, as described herein.

In another aspect of the present invention compositions including a nanostructure comprising gelatin nanoparticles are provided which is accessible to the action of proteases from the pathogen &/or host to allow for digestion of the nanostructure to release the encapsulated drug. The gelatin nanoparticles, thus serves as an alternate substrate for the proteases thereby preventing/minimizing host tissue degradation.

Another aspect of the present invention provides for gelatin matrix for nanostructure development because of its biocompatibility, biodegradability and relatively low antigenicity.

In another aspect, the gelatin matrix has RGD sequences that help in epithelial cell binding and proliferation, while in another aspect, the gelatin matrix may be made to have a positively charged outer surface by conjugating molecules like poly-lysine, chitosan etc.

In another aspect, the nanostructure has RGD sequences. RGD sequences are present abundantly in collagen and aid in cell adhesion and migration. Gelatin, a denatured form of collagen retains this sequence and the presence of these sequences in gelatin has been exploited in various cell attachment studies.

In another aspect of the present invention, the gelatin nanostructure based drug delivery system has RGD sequences and is conjugated to anti-TLR4 (Toll-like receptor) ligands for targeting the corneal epithelium and blocking the inflammatory pathway.

In another aspect, the present invention provides a nanostructure with an increased corneal retention time owing to the presence of RGD repeats and/or anti-TLR ligands.

In yet another aspect, the nanostructure includes one or more ligands chemically conjugated to the surface that may consist of anti TLR ligands either peptides, proteins, antibodies, or any other chemicals which binds and blocks the over expressed TLRs or its associated cell surface molecules and blocks the subsequent inflammatory TLR signaling pathways.

The nanostructure may be conjugated to peptides, carbohydrates, polymers, proteins, antibodies or molecules that have a binding capability to TLRs or its associated molecules and suppress the associated inflammatory response.

In another aspect of the present invention, the nanoparticle comprises a therapeutic agent.

In another aspect of the present invention, the therapeutic agent is one or both, an antimicrobial agent and/or an anti-inflammatory agent.

In yet another aspect of the present invention, the therapeutic agent is an antifungal drug.

In yet another aspect of the present invention, the anti-fungal drug is ketoconazole which has been used as a model drug for treating fungal keratitis.

In yet another aspect of the present invention, ketoconazole has been complexed with methyl-β-cyclodextrin to improve its aqueous solubility.

In another aspect of the present invention ketoconazole has been complexed with methyl-β-cyclodextrin and encapsulated in gelatin matrix.

In another aspect, the nanostructure is about 10-1000 nm in diameter.

There are four main functions to be addressed by the multifunctional gelatin nanoparticles:
1.) They should have an increased residence time at the corneal surface by binding to the corneal epithelial cell surface, either through the integrins (RGD sequences) and/or through the over-expressed TLRs or its associated molecules (Anti-TLR4 antibody),
2.) They should block the TLR signalling pathway thereby suppressing the inflammatory response.
3.) The nanostructure/nanoparticles must degrade, and release the encapsulated drug. The kinetics of the drug release would depend on the protease concentration in the milieu, thus providing an “on demand” drug release.
4.) Anti-microbial effect through the released drug.

Satisfying these criteria simultaneously presented several challenges in nanostructure based drug delivery system design and synthesis.

In another aspect, the present invention provides methods for delivering an anti microbial and/or an anti-inflammatory agent to an infection in a subject. The methods include administering to the subject an effective amount of a nanostructure as described herein.

In another aspect the invention provides a method of encapsulating a hydrophilic drug into the gelatin matrix of the nanostructure. In case of hydrophobic drugs, the drug may be complexed with cyclodextrins (methyl-β-cyclodextrins, hydroxyl propyl β-cyclodextrins etc.) or other molecules that aid in drug dissolution prior to encapsulation in the nanostructure.

In a further aspect, the present invention features methods for treating keratitis (sterile or infectious) in a subject. The methods include administering to the subject a therapeutically effective amount of a nanostructure composition as described herein, thereby treating the subject.

In yet another aspect, the present invention provides methods for inhibiting the growth of a pathogen in a subject and controlling the associated inflammation, the method comprises administering to the subject a therapeutically effective amount of a composition as described herein, there by inhibiting the growth of the pathogen and controlling the associated inflammation in the subject.

In another aspect the invention provides a pharmaceutical composition comprising the novel the nanostructure based drug delivery system described herein and optionally a pharmaceutically acceptable carrier commonly known in the art.

In another aspect, the invention provides use of the compounds and compositions described herein to treat keratitis (sterile or infectious) in a subject.

Accordingly, the main embodiment of the present invention provides a nanostructure based drug delivery system comprising:
(a) gelatin matrix;
(b) anti-TLR4 ligand conjugated to the gelatin matrix surface;
(c) a therapeutic agent;
   wherein
   the gelatin matrix is held together by covalent cross-linking through glutaraldehyde; the anti-TLR4 ligand is an anti-TLR4 antibody that is chemically conjugated to the surface of the nanostructure by EDC-NHS chemistry where the primary amine groups of the anti-TLR4 antibody is conjugated to the free —COOH groups in the gelatin matrix;
   the therapeutic agent is an anti-microbial drug or an anti-inflammatory drug encapsulated in the gelatin matrix.

Another embodiment of the present invention provides a nanostructure based drug delivery system as herein described, wherein the gelatin matrix is made of RGD sequences or a positively charged outer surface by conjugating molecules like poly-lysine, chitosan etc.

Another embodiment of the present invention provides the nanostructure based drug delivery system as herein described, wherein the therapeutic agent is hydrophobic or hydrophilic.

Another embodiment of the present invention provides the nanostructure based drug delivery system as herein described, wherein the hydrophobic therapeutic agent is complexed with a cyclodextrin.

Another embodiment of the present invention provides the nanostructure based drug delivery system as herein described, wherein the cyclodextrin is selected from methyl-β-cyclodextrins, hydroxyl propyl β-cyclodextrins.

Another embodiment of the present invention provides the nanostructure based drug delivery system as herein described wherein the therapeutic agent is ketoconazole.

Another embodiment of the present invention provides the nanostructure based drug delivery system as herein described, wherein the therapeutic agent is released from the nanostructure through degradation by proteases secreted by the host comprising matrix metallo-proteases or serine proteases and/or proteases secreted by the pathogens.

Another embodiment of the present invention provides the nanostructure based drug delivery system as herein described, wherein the nanostructure is about 10-1000 nm in diameter.

Another embodiment of the present invention provides a method of preparing a nanostructure based drug delivery system as herein described, comprising:
a. preparing a solution of the therapeutic agent;
b. dissolving gelatin matrix in the therapeutic agent;
c. preparing drug loaded gelatin nanoparticles using double desolvation method;
d. conjugating anti-TLR4 ligand to the nanoparticles obtained in step (c) using carbodiimide method.

Another embodiment of the present invention provides a method of preparing a nanostructure based drug delivery system as herein described, comprising:
a. dissolution of ketoconazole with methyl-β-cyclodextrin;
b. dissolving of gelatin into ketoconazole-methyl-β-cyclodextrin complex solution obtained in step (a);
c. preparing ketoconazole loaded gelatin nanoparticles using double desolvation method;
d. conjugating anti-TLR4 antibody to the nanoparticles obtained in step (c) using carbodiimide method.

Another embodiment of the present invention provides a method of delivering an antibiotic and/or an anti-inflammatory therapeutic agent to an infection in a subject, wherein the method comprises administering to the subject the nanostructure as herein described.

Another embodiment of the present invention provides a method of treating infectious and/or sterile keratitis, wherein the method comprises administering a therapeutically effective amount of the nanostructure based drug delivery system as herein described.

Another embodiment of the present invention provides use of the nanostructure based drug delivery system as herein described, for treatment of infections or inflammations.

Another embodiment of the present invention provides use of the nanostructure based drug delivery system as herein described, wherein the infection is infectious and/or sterile keratitis.

Another embodiment of the present invention provides a pharmaceutical composition comprising a nanostructure based drug delivery system as herein described.

Another embodiment of the present invention provides use of the pharmaceutical composition as herein described, for treatment of infections or inflammations.

Another embodiment of the present invention provides use of the pharmaceutical composition as herein described, wherein the infection is infectious and/or sterile keratitis.

Another embodiment of the present invention provides a kit for treatment of infectious or sterile keratitis comprising the nanostructure based drug delivery system herein described and an instruction manual.

In the present investigation, gelatin nanoparticles have been synthesized with anti-TLR antibodies conjugated to the surface. The nanoparticles are loaded with anti-fungal drug ketoconazole. The following examples are given by way of illustration and therefore should not be construed to limit the scope of present investigation. Example 1 represents drug dissolution and characterization. Examples 2, 3 and 4 represent synthesis and characterization of the gelatin nanoparticles. Examples 5-8 represent the in vitro evaluation of the nanoparticle system for different activities. Examples 9 and 10 represent the in vivo evaluation of nanoparticles in a rat model of fungal keratitis.

### EXAMPLES

The following examples are given by way of illustration of the present invention and therefore should not be construed to limit the scope of the present invention.

### Example 1: Dissolution of Ketoconazole with methyl-β-cyclodextrin

2.5 mM, 5 mM, 7.5 mM, 10 mM, 15 mM & 20 mM methyl-β-cyclodextrin was used to dissolve an excess amount of ketoconazole in 10 ml milliQ water. The contents of each tube were stirred for 3 days until equilibrium. After equilibration undissolved ketoconazole was separated by filtration with a 0.45 μm PVDF membrane syringe filter. The resulting solution was then analyzed for ketoconazole content by a Lambda Model UV-Visible spectrophotometer (Perkin Elmer, USA) at 260 nm after dilution of the samples.

### Example 2:Particle Synthesis and Anti-TLR4 Antibody Conjugation

Step 1: The double-desolvation method was used for nanoparticle synthesis, partially modified by the method described by Coester et al. 2000. 2.5 g of gelatin type B (Bloom225) was dissolved in 50 ml water (5% w/w) under gentle heating (50° C.). In the first desolvation step 50 ml of acetone was added rapidly to the gelatin solution. After sedimentation of the precipitated gelatin fraction, the supernatant containing soluble low molecular weight gelatin was discarded. The sediment was redissolved again by the addition of 50 ml water under gentle heating (50° C.). The redissolved gelatin containing the high molecular weight fraction was snap freezed in liquid nitrogen and lyophilized. The lyophilized gelatin was stored at 4° C. till further use. 0.1 g of the freeze dried high molecular weight gelatin was dissolved in 10 ml water under gentle heating (50° C.) and the pH was adjusted to 3.5. Nanoparticle formation was initiated by the drop-wise addition of 35 mL acetone (second desolvation step) under continuous stirring (500 rpm). After a few minutes, 20 μl of glutaraldehyde (25%) diluted in 1 ml acetone was added to the reaction mix to crosslink the nanoparticles. After stirring for 12 hours, the particles were purified by three-fold centrifugation (48000×g for 10 min) and redispersion in acetone/water (30/70). The purified nanoparticles were stored as dispersion in MQ water at 4-8° C. or lyophilized.
For the synthesis of drug loaded nanoparticles, gelatin was dissolved in 10 ml of the prepared ketoconazole-methyl-β-cyclodextrin complex (1 mM) solution (before the second desolvation step) to obtain a gelatin (type B) concentration of 1% w/v. The above mentioned protocol for nanoparticle synthesis was followed thereafter.
Step 2: Anti-TLR4 antibody (Abcam) was conjugated to the nanoparticles using carbodiimide chemistry. Briefly, nanoparticles were suspended in 0.1 M MES buffer pH 6 with 0.5 M NaCl. The surface carboxylate functional groups of the nanoparticles were activated by incubation with EDC (2 mM) and NHS (5 mM) at room temperature for 2 hrs with vortexing. After incubation the particles were washed by three fold centrifugation (48,000×g, 10 min) and redispersion in PBS pH 7.4. Antibody solution (2 μg/mg nanoparticles) was added drop-wise to activated nanoparticles and vortexed at 4° C. for 24 hrs. Conjugated nanoparticles were washed by centrifugation (48,000×g, 10 min) and stored at 4° C. till further use.

### Example 3: Particle Size Measurements

Gelatin nanoparticles were resuspended in PBS pH 7.4 and analyzed for their size and polydispersity in a nanopartica nanoparticle analyzer system (Horiba Scientific).

### Example 4: Particle Surface Charge Measurements

For zeta potential measurements gelatin nanoparticles were resuspended in 10 mM phosphate buffer (pH 7.4) at a concentration of 1 mg/ml and the surface charge was analyzed by a nanoparticle analyzer system (Horiba Scientific) at 25° C.

### Example 5: MIC Calculation for Ketoconazole Loaded Gelatin Nanoparticle

Aspergillus flavus (MTCC, CSIR-Imtech) was grown on Sabouraud dextrose agar (SDA) for 7 days at 35° C. After 7 days, 1 ml of sterile PBS was added to the culture and the flask was gently vortexed. The spore suspension was taken out and placed in a sterile tube for a few minutes allowing the heavy particles to settle. The upper homogeneous suspension was transferred to a sterile tube. The densities of the spore suspension was read at 560 nm and adjusted to an optical density (OD) in the range of 0.09 to 0.11 (80 to 82% transmittance). These suspensions were diluted 1:100 in the standard medium which would correspond to 0.4×10⁴ to 5×10⁴ colony forming units (CFU)/ml.

Ketoconazole was diluted and added to different wells of a 96 well plate at increasing concentrations. Similarly, ketoconazole-methyl-β-cyclodextrin complex and gelatin nanoparticles loaded with ketoconazole-methyl-β-cyclodextrin complex were added in various concentrations. The inoculum was incubated at 35° C. for 24 hours after which the contents of each well were plated on SDA plates and incubated at 35° C. Colonies were counted after 2-3 days.

### Example 6: Nanoparticle Interaction with HCE Cells

Human corneal epithelial (HCE) cells (LVPEI, Hyderabad) were cultured in DMEM and Ham's F12 (1:1), supplemented with 5% FBS, 10 ng/ml EGF, 5 μg/ml insulin, antibiotics (5 μg/ml Penicillin & 6 μg/ml streptomycin) at 37° C. under 95% humidity and 5% CO₂. Cells were incubated in serum free media for 24 hours and subsequently treated with 1 μg/ml of LPS. FITC labeled anti-TLR4 conjugated gelatin nanoparticles and bare gelatin nanoparticles were added at a concentration of 0.2 mg/ml (either to LPS treated or untreated HCE cells) and incubated for 4 hours. Cells were washed with PBS, fixed with 4% formaldehyde solution and counter-stained with DAPI. Untreated cells served as negative control. Images were taken on a confocal microscope using a 63× objective lens on a Leica LAS-AF-TCS-SP8. The images were analyzed by LAS AF software provided by the company.

For flow cytometry analysis, cells were treated as mentioned previously. Post treatment cells were scraped and nanoparticle interaction was studied by flow cytometry using a BD FACSCalibur™ cell analyzer and analysis was done by Cell Quest software (Becton Dickinson).

### Example 7: RT-PCR to Study the Anti-Inflammatory Effect of the Nanoparticles

For RT-PCR studies, cells were cultured in serum free media followed by treatment with either 100 ng/ml LPS, 100 ng/ml LPS along with 5 mg GNP and 100 ng/ml LPS along with 5 mg anti-TLR4 conjugated GNP. Untreated cells served as normal controls. Cells were collected after 4 hours of treatment and resuspended in Trizol (Life Technologies). The total RNA isolated was given a DNase I (NEB) treatment according to the manufacturer's instructions. 1 μg of DNase I treated RNA was taken for cDNA synthesis using SuperScript® First-Strand Synthesis kit for RT-PCR as per instructions. For quantitation of genes, 1 μl of cDNA was used as a template with the corresponding primers (sequences provided in Patent In software separately) for the genes to be studied. The polymerase chain reaction (PCR) products were analyzed by running on a 2% agarose gel in Tris-Acetate-EDTA (TAE) buffer. The resulting bands were documented by a Gene Genius Classic Gel Documentation System, Syngene, UK using the GeneSnap software. Densitometric scans and comparative calculations were done using Image J software (CSHL). The primers sequences used for PCR are given in the table.

### Example 8: Drug Release from the Gelatin Nanoparticles

100 mg GNPs were resuspended in PBS with varying concentrations of Pronase (0 mg/ml, 0.01 mg/ml & 0.1 mg/ml) and packed in dialysis tubings MWCO 8 K to 12 K. Samples were collected at varying time points and analyzed by using reverse-phase high performance liquid chromatography (RP-HPLC) for drug content.

Ketoconazole was analysed using an Agilent 1200 Series reverse-phase high performance liquid chromatography (RP-HPLC) system. Separation was carried out using an Agilent 300SB-C18 (5 μm, 4.5×250 mm) column. Isocratic elution was carried with the mobile phase consisting of acetonitrile and 0.2% triethylamine pH adjusted to 6.4 with phosphoric acid (48:52, v/v) at flow rate of 1 mL/min. The mobile phase was filtered under vacuum and degassed. Chromatographic separation was monitored at 260 nm. All the samples were analyzed at room temperature. Total run time for the analysis was 20 minutes.

### Example 9: Induction of Fungal (Aspergillus flavus) Keratitis

24 wistar rats (Animal house, CSIR-CCMB) weighing 200 to 250 grams each were randomly distributed into 6 groups of four rats each. All rats were treated in accordance with the Association for Research in Vision and Ophthalmology (ARVO) statement for the use of animals in ophthalmic vision and research. The protocol was approved by the institutional animal ethics committee (IAEC).

The rats were anaesthetized for the duration of the experiment with intra-peritoneal injection of ketamine hydrochloride. Rats were administered with subcutaneous injection of prednisolone prior to inoculation of fungal spores in order to establish the infection. Rats in all groups received 20 μl of the spore suspension (10⁸ CFU/ml) in their right eye. The left eye in each rat served as an un-inoculated control. Post inoculation rats were examined daily for 7-8 days. Rats that developed keratitis (characterized by a white infiltrate) within 48 hours post inoculation were considered for the testing of different formulations as follows,

Group 1: untreated,
Group 2: administered with PBS,
Group 3: administered with ketoconazole,
Group 4: administered with empty gelatin nanoparticles,
Group 5: administered with ketoconazole-methyl-β-cyclodextrin complex encapsulated gelatin nanoparticle, and
Group 6: administered with Anti-TLR4 conjugated ketoconazole-methyl-β-cyclodextrin complex encapsulated gelatin nanoparticle.
Following inoculation of Aspergillus suspensions into the rat corneas, the eye was examined using a slit-lamp bio-microscope till 7 days.

### Example 10: Corneal Sample Collection and Processing

Rats in all groups were sacrificed on the 7^{th} or 8^{th} day of treatment by carbon-dioxide inhalation. Eye balls were excised and taken for either microbiological or histo-pathological evaluation.

### Microbiological Evaluation

The eye ball was excised from the eye socket under sterile conditions. The entire eye ball was inoculated on a SDA plate and incubated at 35° C. for 7 days. The plates were examined daily for 7 days for the growth of Aspergillus flavus.

### Histopathological Evaluation

The eye ball was excised from the eye socket and fixed in 10% formalin. Cornea was isolated and embedded in paraffin wax for sectioning. The paraffin sections were subjected to staining by Haematoxylin and Eosin (H & E) stain for the visualization of tissue integrity and infiltration of inflammatory cells.

### Advantages

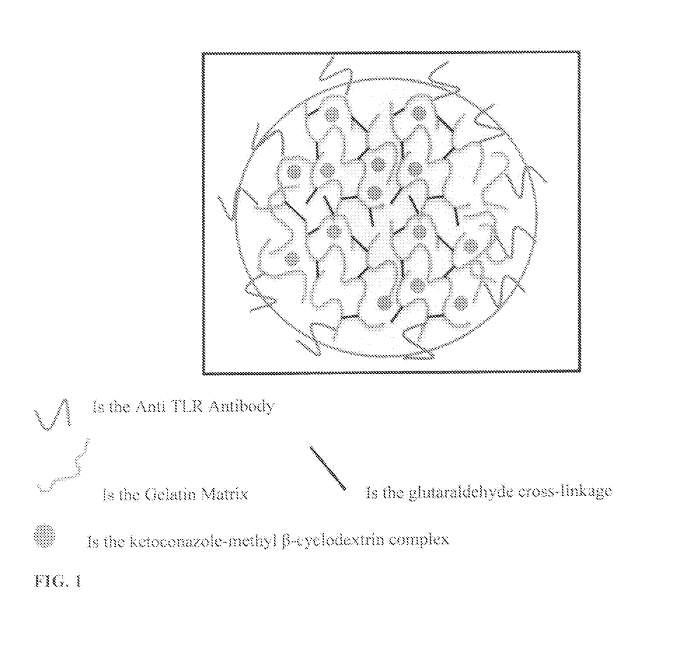

The gelatin nanoparticles used for the present invention fulfills all the shortcomings of the current practices for keratitis management. The system has been designed and developed in a way that the desired features (corneal binding, anti-microbial property & anti-inflammatory properties) come into effect after a host stimuli. The main advantages of this system include:
1. The nanoparticle formulation provided herein and represented in FIG. 1 is novel.
2. Their synthetic methodology is very economical.
3. The nanoparticle formulation represented in FIG. 1 has a biocompatible and biodegradable gelatin core/matrix, susceptible to degradation and drug release in the infection and inflammatory environment.
4. The nanoparticle formulation represented in FIG. 1 has an antifungal drug ketoconazole encapsulated in its matrix that can be released by simple diffusion or with the degradation of the gelatin matrix.
5. The nanoparticle formulation represented in FIG. 1 is conjugated to anti-TLR4 antibodies which serve a dual purpose of corneal adhesion and suppression of inflammation. Anti-TLR4 antibodies are used as a proof of concept study and can be replaced by more economical synthetic anti-TLR4 ligands.
6. The nanoparticle formulation represented in FIG. 1 binds to the corneal surface and suppresses inflammation. The binding and suppression is dependent on the severity of the infection and associated inflammation. Thus, the particle binding and its subsequent effects are placed under a host trigger system. Such a regulation is advantageous in a condition like keratitis where the use of anti-inflammatory drugs is a point of debate.
7. The nanoparticle formulation represented in FIG. 1 releases the drug ketoconazole in response to the protease concentration in the microenvironment of the infection. Such a regulated drug release is advantageous in a condition like keratitis where the drug dosage is frequent and decided according to the severity of infection.
8. The nanoparticle formulation represented in FIG. 1 can act as an alternative substrate for the proteases present in the local microenvironment of the infection & inflammation, thereby preventing/minimizing host corneal damage.

### REFERENCES

1. Akpek and Gottsch 2003

2. Bailey and Berkland 2009

3. Coester et al. 2000.

4. Conway 2008

5. Dajcs et al. 2004

6. Dvir et al. 2010

7. Fukata et al. 2006

8. Gao et al. 2011

9. Ghannoum and Rice 1999

10. Gokhale 2008

11. Guo et al. 2012.

12. Haurowitz et al. 1943

13. Ibeagha-Awemu et al. 2008

14. Johnson and Pearlman 2005

15. Johnson et al. 2005

16. Klotz et al. 2000

17. Knop and Knop 2005a

18. Knop and Knop 2005b

19. Kompella et al. 2010

20. Kumar and Yu 2006

21. Kumar et al. 2006

22. Loftsson and Stefansson 2002.

23. Lu et al. 2009

24. Madianos et al. 2005

25. Monod et al. 2002.

26. Mura et al. 2013

27. Ofokansi et al. 2010

28. Patel et al. 2012

29. Pierschbacher and Ruoslahti 1984

30. Pierschbacher and Ruoslahti 1984b

31. Ren and Wu 2011

32. Salvati et al. 2013

33. Song et al. 2007

34. Thibodeaux et al. 2004

35. Vandervoort and Ludwig 2004

36. Vantrappen et al. 1985

37. Wang et al. 2011

38. Wilhelmus 2002

39. Zhang et al. 2008

40. Zhao and Wu 2008a

41. Zhao and Wu 2008b

42. Zhu et al. 2012.

1. A nanostructure based drug delivery system comprising:
(a) gelatin matrix;
(b) anti-TLR4 ligand conjugated to the gelatin matrix;
(c) a therapeutic agent;
wherein
the gelatin matrix is held together by covalent cross-linking through glutaraldehyde;
the anti-TLR4 ligand is an anti-TLR4 antibody that is chemically conjugated to the surface of the nanostructure by EDC-NHS chemistry where the primary amine groups of the anti-TLR4 antibody is conjugated to the free —COOH groups in the gelatin matrix;
the therapeutic agent is an anti-microbial drug or an anti-inflammatory drug encapsulated in the gelatin matrix.

2. The nanostructure based drug delivery system as claimed in claim 1, wherein the gelatin matrix is made of RGD sequences or a positively charged outer surface by conjugating molecules like poly-lysine, chitosan etc.

3. The nanostructure based drug delivery system as claimed in claim 1, wherein the therapeutic agent is hydrophobic or hydrophilic.

4. The nanostructure based drug delivery system as claimed in claim 1, wherein the hydrophobic therapeutic agent is complexed with a cyclodextrin.

5. The nanostructure based drug delivery system as claimed in claim 1, wherein the cyclodextrin is selected from methyl-β-cyclodextrins, hydroxyl propyl β-cyclodextrins.

6. The nanostructure based drug delivery system as claimed in claim 1, wherein the therapeutic agent is ketoconazole.

7. The nanostructure based drug delivery system as claimed in claim 1, wherein the therapeutic agent is released from the nanostructure through degradation by proteases secreted by the host comprising matrix metallo-proteases or serine proteases and/or proteases secreted by the pathogens.

8. The nanostructure based drug delivery system as claimed in claim 1, wherein the nanostructure is about 10-1000 nm in diameter.

9. A method of preparing a nanostructure based drug delivery system as claimed in claim 1, comprising:
a. preparing a solution of the therapeutic agent;
b. dissolving gelatin matrix in the therapeutic agent;
c. preparing drug loaded gelatin nanoparticles using double desolvation method;
d. conjugating anti-TLR4 ligand to the nanoparticles obtained in step (c) using carbodiimide method.

10. The method of preparing a nanostructure based drug delivery system as claimed in claim 1, comprising:
a. dissolution of ketoconazole with methyl-β-cyclodextrin;
b. dissolving of gelatin into ketoconazole-methyl-β-cyclodextrin complex solution obtained in step (a);
c. preparing ketoconazole loaded gelatin nanoparticles using double desolvation method;
d. conjugating anti-TLR4 antibody to the nanoparticles obtained in step (c) using carbodiimide method.

11. A method of delivering an antibiotic and/or an anti-inflammatory therapeutic agent to an infection in a subject, wherein the method comprises administering to the subject the nanostructure of claim 1.

12. A method of treating infectious and/or sterile keratitis, wherein the method comprises administering a therapeutically effective amount of the nanostructure based drug delivery system as claimed in claim 1.

13. Use of the nanostructure based drug delivery system as claimed in claim 1, for treatment of infections or inflammations.

14. Use of the nanostructure based drug delivery system as claimed in claim 1, wherein the infection is infectious and/or sterile keratitis.

15. A pharmaceutical composition comprising a nanostructure based drug delivery system as claimed in claim 1.

16. Use of the pharmaceutical composition as claimed in claim 15, for treatment of infections or inflammations.

17. Use of the pharmaceutical composition as claimed in claim 15, wherein the infection is infectious and/or sterile keratitis.

18. A kit for treatment of infectious or sterile keratitis comprising the nanostructure based drug delivery system as claimed in claim 1 and an instruction manual.
